(19) United States
(12) Patent Application Publication
(10) Pub. No.: US 2010/0047324 A1
(43) Pub. Date: Feb. 25, 2010

(54) Multi-Functional Wound Dressing Matrices and Related Methods

(75) Inventors: Ulf Fritz, Hirschhorn (DE); Olaf Fritz, Hirschhorn (DE); Thomas A. Gordy, Newnan, GA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: CeloNova BioSciences, Inc., Newnan, GA (US)

(21) Appl. No.: 12/391,040

(22) Filed: Feb. 23, 2009

Related U.S. Application Data
(60) Provisional application No. 61/030,707, filed on Feb. 22, 2008.

(51) Int. Cl. A61L 15/16; A61P 17/02

(52) U.S. Cl. 424/446

(57) ABSTRACT

Various embodiments are directed to multi-functional wound-care dressing matrices that can protect and promote new tissue growth at a wound site. The multi-functional wound care matrix can incorporate polyphosphazenes of formula I, as a component that can be configured into various forms, including as fibrous mats, porous membranes, nonporous films, particulate formulations, and equivalents. The multi-functional wound-care dressing matrix of the present disclosure exhibit high-performance properties conferred by polyphosphazenes of formula I. Exceptional biocompatible properties of polyphosphazenes of formula I provide an ideal tissue-contacting surface for the multi-functional wound-care dressing matrix of interest.

### CROSS-REFERENCE TO RELATED APPLICATIONS

This application claims the benefit under 35 U.S.C. §119(e) of U.S. Provisional Patent Application No. 61/030,707, filed Feb. 22, 2008, incorporated herein by reference in its entirety.

### TECHNICAL FIELD

This disclosure relates to various articles/devices, incorporating and/or encapsulated by polyphosphazene polymers, that can enhance the care and treatment of various types of wounds, and related methods.

### BACKGROUND

Various articles and devices have been developed to manage the care and treatment of tissue injury or wounds. Wounds are susceptible to many secondary effects that occur after the initial tissue injury, including further mechanical trauma, pathogenic infiltration, infection, dehydration, excessive fluid discharge, sepsis, inflammation, pus formation, scar tissue formation, hardening of healthy tissue and/or tissue necrosis. The selection of an appropriate wound dressing to suit a specific type of tissue damage at a wound site can significantly promote the healing process with reduced secondary effects, including scar formation and pain. Many dressings can adhere to the surface of delicate de novo epidermal layer, and can result in extensive tissue scarring when frequent changes in dressings may be required. Insufficient wound care can significantly reduce the healing rate, promote other secondary complications such as infections, and induce additional discomfort and pain. High-performance wound dressings that can exhibit multi-functional properties are highly desirable to treat various types of human and animal wounds.

### SUMMARY OF THE INVENTION

Various embodiments are directed to multi-functional wound-care dressing matrices (“MFWDM”) that can protect and promote new tissue growth at a wound site. The multi-functional wound-care dressing matrix can incorporate polyphosphazenes of formula I, as a component that can be configured into various forms, including as fibrous/non-fibrous mats, porous/non-porous membranes, porous/non-porous films, open-cell/closed-cell foams, particulate formulations for spray-on applications, the equivalent of these forms, and combinations thereof. The polyphosphazenes of formula I exhibit a broad range of unique chemical and physical properties that can be incorporated into a multitude of wound care products contemplated in this disclosure as multi-functional wound-care dressing matrices (“MFWDM”): as a primary structural component, a coating layer to encapsulate another structural component, and/or a mediator component to support various non-structural functionalities. The incorporation of polyphosphazenes of formula I into the construction of MFWDM of interest can provide substantial advantages to promote optimal healing at a given wound site.

### BRIEF DESCRIPTION OF THE SEVERAL VIEWS OF THE DRAWINGS

FIG. 1 is a schematic of a sheet of hypothetical substrate layer that can be incorporated into a multi-functional wound-care dressing matrix, as one embodiment of the present disclosure.

FIG. 2 is a schematic of a multi-functional wound-care dressing matrix comprising multiple substrate layers, as one embodiment of the present disclosure.

FIG. 3 is a schematic of a multi-functional wound-care dressing matrix comprising multiple substrate layers, and includes capsules, as one embodiment of the present disclosure.

FIG. 4 is a schematic of a multi-functional wound-care dressing matrix comprising multiple substrate layers, and includes polymers of formula I formulated as a foam/sponge, as one embodiment of the present disclosure.

FIG. 5 is a schematic of a multi-functional wound-care dressing matrix comprising multiple substrate layers, and formulated as a adhesive wound patch, as one embodiment of the present disclosure.

### DETAILED DESCRIPTION OF DISCLOSURE

### A. Definitions

In addition to the definition of terms provided below, the terms “a” or “an” can mean one or more of the referenced subject matter.

The term “substrate layer” includes any material, including various natural materials, synthetic polymer materials, and combinations thereof. In various embodiments, the substrate layer incorporates polymers of formula I. In various embodiments, the substrate layer is encapsulated, partially or entirely, by polymers of formula I. In other embodiments, the substrate layer incorporating polymers of formula I includes a tissue-contacting surface. In other embodiments, the substrate layer can be formed in situ when a formulation of polymers of formula I can be sprayed onto a wound site in order to form a tissue-surface contacting film. In various embodiments, the substrate layer can be pre-formed into any shape of interest into any two-dimensional and three-dimensional forms. One or more substrate layers can be vertically layered, or stacked, or otherwise favorably combined, blended, or mixed to produce a “multi-functional wound-care dressing matrix” (“MFWDM”).

The term “multi-functional wound-care dressing matrix/matrices” (“MFWDM”) comprising polymers of formula I intended to contact a tissue-surface of a wound site in order to provide multi-functional properties that can promote healing of injured tissue and provide a protective physical barrier. In an embodiment, the MFWDM can be formed in situ when a formulation of polymers of formula I can be sprayed onto a wound site in order to form a tissue-surface contacting film. In various embodiments, the MFWDM can be pre-formed into any shape of interest into any two-dimensional and three-dimensional forms. The MFWDM can be formed as a woven fabric layer, a non-woven fabric layer, a porous film, a non-porous film, a porous membrane, a non-porous membrane, an open-cell foam, a closed-cell foam, a woven mat, a non-woven mat, a mesh, a pad, a sponge, a foam, a gauze, or equivalents, and/or combinations thereof, known by persons skilled in the art. The MFWDM can be produced to include multiple layers of pre-formed layers, in which each pre-formed layer serve various functions including: to absorb excess fluids, to release moisturizers, to provide various agents of interest, to provide mechanical strength, to prevent loss of moisture, to promote collagen formation, to promote tissue regeneration. The MFWDM can be secured to a wound site by any means, including taping, fastening, and/or employing any adhesive known to persons skilled in the art. Embodiments of MFWDM include products that can be substituted for other types of wound dressings, surgical dressings, compression dressings, band-aids, compression bandages, wound meshes, wound drapes, wound scaffolds, surgical fabric adhesives/tapes, medical grade gauzes, medical grade pads, medical grade sponges, burn dressings, or equivalents, and/or combinations thereof, known by persons skilled in the art.

The term “wounds” refers to any injury resulting in tissue damage, tissue penetration, laceration, or lesions, and includes injury induced by various cosmetic treatments. The wounds amenable to treatment by MFWDM include injuries that can be located in any site, including internal, interfacial, external, interstitial, extracorporeal, and/or intracorporeal. Examples of wounds suitable for coverage with the disclosed MFWDM include: cuts, gashes, open wounds, tissue rupture, Decubitus, Dermatitis, lesions, chronic wounds, battlefield wounds, necrotic wounds, acute, chronic, traumatic, lacerations, abrasions, contusions, necrotizing facitis, toxic epidermal nercolysis, pressure wounds, venous insufficiency ulcers, arterial ulcers, diabetic or neuropathic ulcers, pressure ulcers, mixed ulcers, burn wounds, Mucormycosis, Vasculitic wounds, Pyoderma, gangrenosum, and equivalents, and/or combinations thereof, known by persons skilled in the art. Treatment of wounds in human and animal subjects are contemplated by the disclosed MFWDM.

The term “tissue-contacting surface” refers to at least one surface of a multi-functional wound care matrix of interest intended to make contact with the wound site.

The term “substrate layer” refers on the individual layers composing the MFWDM. However, if the MFWDM comprises two or more layers, then the substrate layer in direct contact with the wound site has the tissue-contacting surface, and the other substrate layers are positioned above the substrate layer (“superimposed substrate layer”) in closest proximity to the wound site.

The term “tissue surface” includes internal, interfacial, interstitial, or external surface(s) of human and animal bodies, such as, but not limited to vessels, organs, skin, cavities, bones, cartilages, or other equivalents.

The term “incorporating” refers to the structural integration of polymers of formula I into a suitable MFWDM of interest, in which the polyphosphazene polymers can be incorporated as components of fibers, films, membranes, meshes, sieves, mats, or equivalents known to persons skilled in the art, and/or combinations thereof.

The terms “encapsulating” and “coating” and “blending” can be used interchangeably to refer to an enclosure of a substrate layer(s), partially or entirely, by employing various polymers of general formula I. The MFWDM is not limited as to the exact disposition of the polyphosphazene matrix, for example, the polyphosphazene matrix can be coated (or layered) with, reacted with, blended (or mixed) with, embedded, grafted to, bonded to, crosslinked with, copolymerized with, coated and/or reacted with an intermediate layer that is coated and/or reacted with, or combined with other conventional biomaterials in any manner. Further, the polyphosphazene can be combined with a conventional biomaterial, and the combination can be coated on a device or a surface such that the polyphosphazene and biomaterial are coated at substantially the same time. All these aspects are encompassed by the disclosure that any material includes or comprises a biomaterial and a polyphosphazene, or by the disclosure that a polyphosphazene is added to a biomaterial or medical device.

The term “protective barrier” refers to any physical barrier that prevents viral, microbial, fungal infection; prevents further physical damage; prevents loss of fluid from exposed tissue surfaces; protects from extreme environmental conditions, including extreme heat and cold temperatures; protects from the entry of environmental water into the wound; promotes healing; prevents scarring; reduces pain; reduces inflammation; reduces bleeding; promotes blood clotting; prevents adhesion to wound surface; promotes de novo collagen formation; promotes tissue regeneration; promotes innervation; promotes vascularization; decreases the period for healing, and/or promotes cellular growth rates.

The term “film(s)” refers to any two-dimensional matrix composed of any material, including polymers of formula I that can be produced by any methods known to persons skilled in the art.

The term “fluid(s)” or “liquid(s)” can be interchangeably used in reference to a contacting matter, includes common liquids, semi-solids, pastes, sols or gels, such as pharmaceutical ointments, that may contain a considerable amount of extractable liquid(s).

The term “foam(s)” refers to any three-dimensional matrix composed of any material, including polymers of formula I that can be produced by any methods known to persons skilled in the art.

The term “spray(s)” refers to any pressurized aerosol dispenser that can be employed to deploy particulates of polymers of formula I in order to deposit in situ the polymers on top of a target wound site.

The terms “carrier member(s)” or “capsules” can interchangeably refer to particles composed mainly of natural and/or synthetic polymers of any shape or surface contour having an average diameter size ranging from approximately 10 μm to approximately 1200 μm. A carrier member can include any molecule of interest, including growth factors, peptides, proteins, hormones, carbohydrates, polysaccharides, nucleic acids, lipids, vitamins, steroids, antibiotics, anti-inflammatory, and organic or inorganic drugs.

The term “decontaminants” include antiseptic agents, such as ubck Chlorhexidine gluconate, Methylisothiazolone, Thymol, .alpha.-Terpineol, Cetylpyridinium chloride, Chloroxylenol, or equivalents known to persons skilled in the art, and/or combinations thereof.

The term “agents of interest” include various types of decontaminants, healing agents, exudate absorbers, anti-microbial, anti-viral, anti-fungal, anti-scarring agents, anti-histamine, non-steroidal anti-inflammatory agents, antithrombotic agents, or equivalents and/or combinations thereof, known to persons skilled in the art.

The term “healing agents” include one or more drugs, bioactive agents, nutraceuticals, or equivalents known to persons skilled in the art, and/or combinations thereof.

The term “anti-microbial” refers to any naturally or synthetic entity that can reduce microbial levels: Penicillin; Penicillin G, Penicillin V, erythromycin, lincomycin, clindamycin, novibiocin, vancomycin, fusidic acid, rifampicin, polymyxins, neomycin, kanamycin, tobramycin gentamycin, amoxicillin, ampicillin, azlocillin sodium, dicloxacillin sodium, furoxacillin, mecillinam, Beta-lactamase resistant penicillin; Methicillin, Nafcillin, Oxacillin, Cloxacillin; novobiocin; leucomycins, josamycin, maridomycin, midecamycin, spiramycin; lincomycins, clindamycin, linocmycin; macrolides, rosamycin; penicillins, Extended spectrum penicillin; Ampicillin, Amoxicillin, Carbenicillin, Ticarcillin, Piperacillin, Drugs given in combination with penicillin (beta-lactamase inhibitors); Clavulanic acid, Sulbactam, Tazobactam; Cephalosporins; Cephalothin, Cefazolin, Cephalexin, Cephradine; Cefamandole; Cefaclor, Cefuroxime, Cefonicid, Cefoxitin, Cefotetan, Cefotaxime, Ceftazidime, Cefoperazone, Ceftizoxime, Ceftriaxone, Cefixime, Cefepime, Imipenem, Meropenem, Monobactam, Aztreonam, Vancomycin, Cycloserine, Bacitracin, Fosfomycin, Aminoglycosides; Streptomycin, Neomycin, Gentamicin, Tobramycin, Amikacin, Netilmicin, butirosin, didesoxykanamycin B (DKB), fortimycin, gentamycin, kanamycin, lividomycin, ribostamycin, sagamycines, seldomycins and their epimers, sisomycin, sorbistin, tobramycin; Tetracycline's; Tetracycline, Oxytetracycline, Demeclocycline, Minocycline, Doxycycline, Macrolides; Erythromycin, Clarithromycin, Azithromycin, Clindamycin, Streptogramins, Quinupristin-dalfopristin, Linezolid, Chloramphenicol, DNA synthesis inhibitors; Sulfonamides, Sulfadiazine, Sulfacetamide, Sulfamethoxazole, Sulfadoxine, Sulfasalazin, Trimethoprim, Fluoroquinolones; Ciprofloxacin, Ofloxacin, Lomefloxacin, Norfloxacin, and Enoxacin.

The term “anti-fungal” refers to any naturally or synthetic entity that can reduce microfungal levels, such as Azoles; Ketoconazole, Miconazole, Clotrimazole, Fluconazole, Itraconazole, Allylamines; Terbinafine, Naftifine, Amphotericin B, Nystatin, Flucytosine, Griseofulvin, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione, and octopirox.

The term “antiviral” refers to any naturally or synthetic entity that can reduce microbial levels, such as Tricyclic amines; Rimantidine, Amantidine, Neuraminidase Inhibitors; Oseltamivir, Zanamivir, Nucleoside Analogs; Acyclovir, Valacyclovir, Famciclovir, Penciclovir, Trifluridine, Vidarabine, Ganciclovir, Valaganciclovir, Cidofovir, Pyrophosphanate; Foscarnet, Guanosine Analogs; Ribovarin, Glycoproteins; Interferon-alfa, and interferon-beta.

The term “local anesthetic agents” refer to any naturally or synthetic entity that can induce anesthesia, or reversible depress neuronal function, producing total or partial loss of pain sensation, such as Tetracaine, Cocaine, Procaine, Novocain, benzocaine, bupivacaine, Marcaine, ropivacaine, Naropin, Etidocaine, Duranest, lidocaine, Xylocalne, Prilocalne, Citanest, Mepivacaine, Carbocaine, and Isocaine.

The term “anti-scarring agents” refer to any naturally or synthetic entity that can reduce scar formation, such as Dipyridamole, Amoxapine, Paroxetine, Prednisolone, Dipyridamole, Dexamethasone, Econazole, Diflorasone, Alprostadil, Amoxapine, Ibudilast, Nortriptyline, Loratadine, Albendazole, Pentamidine, Itraconazole, Lovastatin, Terbinafine, and steroids.

The term “anti-histamine” includes any naturally or synthetic entity that can reduce histamine levels: antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like;

The term “non-steroidal anti-inflammatory agents (NSAIDs)” refers to any naturally or synthetic entity that can reduce inflammation, such as propionic acid derivatives (e.g., aminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, aldlofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone) and cyclooxygenase-2 (COX-2) inhibitors such as celecoxib (Celebrex®) and rofecoxib (Vioxx®)

The term “opioid analgesics” includes codeine, fentanyl, hydromorphone, levorphanol, meperidine, methadone, morphine, oxycodone, oxymorphone, propoxyphene, buprenorphine, butorphanol, dezocine, nalbuphine, pentazocine, Fentanyl, Sublimaze, Sufentanil, Sufenta, Alfentanil, Alfenta, Remifentanil, Ultiva, Meperidine, Demerol, Methadone, Dolophine, Morphine, Hydromorphone, Dilaudid, oxymorphone, numorphan, Codeine with acetaminophen, aspirin, Oxycodone with acetaminophen, Percocet, Percodan, dihydrocodiene, hydrocodone with acetaminophen, Vicodin with ibuprofen, Propoxyphene Levorphanol, Levo-Dromoran, Butorphanol, Stadol, Buprenorphine, Buprenex, Nalbuphine, Nubain, Pentazocine, Talwin, Dezocine, Dalgan, Naloxone, Narcan, Naltrexone, Re Via, Depade, Nalmefene, Revex, Diphenoxylate, Loperamide, Imodium, and Dextromethorphan, as well as synthetic or naturally endorphine acting substances.

The term anti-thrombotic agents include thrombolytic agents; streptokinase, alteplase, anistreplase, reteplase, heparin, hirudin, warfarin derivatives, .beta.-blockers, atenolol, beta-adrenergic agonists, isoproterenol, ACE inhibitors, vasodilators, sodium nitroprusside, nicardipine hydrochloride, nitroglycerin, and enalaprilat.

The term “exudate absorber” refers to any substance that can absorb excess fluids discharged from an injured tissue site, and can be produced in any form or shape including, as a flat sheet, beads, pastes, powders, flakes, or equivalents, and/or combinations thereof.

### B. Multi-Functional Wound-Care Dressing Matrices Incorporating Polyphosphazenes of Formula I

1. An Overview of Multi-Functional Wound-Care Dressing Matrices Contemplated

In various embodiments, the multi-functional wound-care dressing matrices (“MFWDM”) of the present disclosure can protect and promote new tissue growth at a wound site. The MFWDM contemplated exhibit properties that enable the management of wound care by protecting injured tissue in a nurturing environment and proactively providing other tissue-regeneration promoting factors to promote the healing rate at a given wound site. Advanced properties of polyphosphazenes of formula I enable prolonged exposure time to various biological fluids and delicate tissues, if desired.

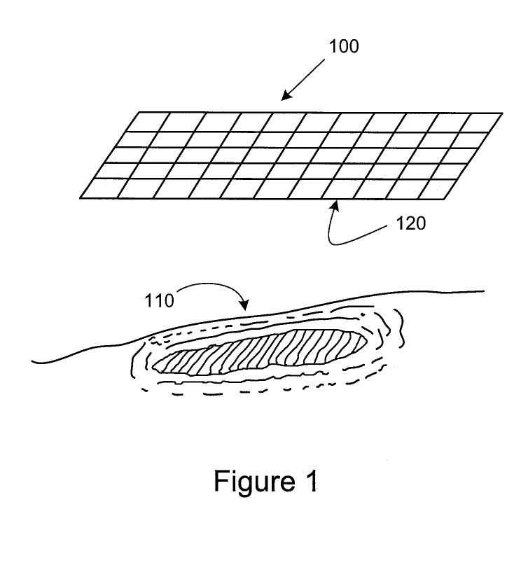

FIG. 1 is a schematic of a sheet of hypothetical substrate layer that can be incorporated into a multi-functional wound-care matrix, as one embodiment of the present disclosure. In FIG. 1, a sheet of an exemplary substrate layer 100 is shown, representing a foundational layer for constructing a multi-functional wound-care dressing matrix. Suitable materials for producing a substrate layer 100 includes any synthetic polymers, polymer blends, and naturally occurring organic or inorganic materials derived from plant, mineral or animal sources. The sheet of a substrate layer 100 of interest can be cut into any multitude of shapes, squares, circles, ellipses, half-moon shape, rectangles, and others. Suitable thickness of the substrate layer can range from about 10 μm up to about 1 cm, from about 10 μm up to about 80 mm, from about 10 μm up to about 60 mm, from about 10 μm up to about 50 mm, from about 10 μm up to about 40 mm, from about 10 μm up to about 30 mm, from about 10 μm up to about 20 mm, from about 10 μm up to about 10 mm, from about 10 μm up to about 5 mm, and/or from about 10 μm up to about 1 mm. One or more hypothetical substrate layers can be vertically assembled, or stacked, to produce a multi-functional wound-care dressing matrix of interest. The MFWDM can be placed over a wound site 110 to provide a protective physical barrier during the healing process. When positioned over a wound site 110, the tissue-contacting surface 120 of the MFWDM makes direct, or indirect, contact with the bodily fluids, such as blood or exudate, and/or cellular tissue matter.

Many protective barrier materials, such as sterile wound dressings, drainage materials, pads, patches, band aids, gauze, foams, sponges and so forth, can be manufactured or derived from a combination of modified natural products and synthetic polymers because the resultant composite materials exhibit advantages, including physical and mechanoelastical properties and/or manufacturing and processing control over the desired shapes produced. Examples of synthetic or natural polymeric biomaterials that can be incorporated as a suitable substrate layer for producing the multi-functional wound-care dressing matrix, include, but are not limited to, polyurethanes, polycarbonates, polyesters, polyamides, polyimides, polyvinyls, polyolefins, Teflon™, Gore-Tex™, polyvinyl alcohols, polyethyleneoxides, polyacrylates, -methacrylates and -cyanoacrylates, latex, polyvinyl chlorides, polylactic and polyglycolic acid derivatives, hydrogel forming agents such as PHEMA, polyethylene oxides, hyaluronic acid, chitosan, alginate, cellulose, and other equivalents known to persons skilled in the art. Each natural or synthetic fibers composing the substrate layer of interest can be formed as individually spun fibers, as fiber bundles, as twisted cables, as wovens, as nonwovens, as knitted, as knotted, or any equivalents, and any combinations thereof.

In various embodiments, the suitable substrate layer for producing the multi-functional wound-care dressing matrix comprises at least one polymer of general formula (I). In various embodiments, the suitable substrate layer for producing the multi-functional wound-care dressing matrix comprises poly[bis(trifluoroethoxy)polyphosphazene] and/or derivatives thereof.

In various embodiments, the multi-functional wound-care dressing matrices can incorporate polyphosphazenes of formula I (defined below), as a component that can be configured into various forms, including as fibrous/nonfibrous mats, porous/non-porous membranes, porous/non-porous films, open-cell/closed-cell foams, particulate formulations for sprayed-on applications, the equivalent of these forms, and combinations thereof.

In various embodiments, the multi-functional wound-care dressing matrices can exhibit exceptional properties inherent to polyphosphazenes by incorporating polyphosphazenes of formula I as a component, for example, as a primary structural component, as a coating layer to encapsulate another structural component, and/or as a mediator component to support various non-structural functionalities.

In various embodiments, the multi-functional wound-care dressing matrices can incorporate polyphosphazenes of formula I as a structural component of the MFWDM, such as 100, wherein one surface of the structural component can function as a tissue-contacting surface.

In various embodiments, the multi-functional wound-care dressing matrices can incorporate polyphosphazenes of formula I as a coating layer of a structural component of the MFWDM, such as 100, wherein the coating layer encapsulating the structural component can function at least as a tissue-contacting surface.

In various embodiments, the multi-functional wound-care dressing matrices can incorporate polyphosphazenes of formula I as a mediator component, wherein the mediator component can function to provide a multitude of functionalities, including as a carrier member capable of storing various agents of interest, such as bioactive agents, pharmaceutical compositions, neutraceuticals, and other equivalents that promote tissue healing and tissue regeneration, known to persons skilled in the art. In various embodiments, the MFWDM of formula I can function as an intermediate MFWDM interfacial, or external surface surface component of the device.

2. Advantages and High-Performance Properties of Multi-Functional Wound-Care Dressing Matrices

In various embodiments, the disclosed multi-functional wound-care dressing matrices incorporating polyphosphazenes of formula I as a component, can exhibit superior bio- and hemocompatibility properties when compared to other polymeric materials conventionally employed as biomaterials. The incorporation of polyphosphazenes of formula I as a component of the disclosed MFWDM of interest can significantly reduce thrombogenicity and platelet adhesion, and thus, can be particularly well-suited as a blood-contacting and/or soft-tissue contacting component of various MFWDM contemplated.

In various embodiments, the disclosed multi-functional wound-care dressing matrices incorporating polyphosphazenes of formula I as a component, can exhibit anti-inflammatory properties. The incorporation of polyphosphazenes of formula I as a component of the disclosed MFWDM of interest can significantly reduce inflammation of a given wound site. In various embodiments, the disclosed multi-functional wound-care dressing matrices incorporating polyphosphazenes of formula I as a component, can exhibit anti-bacterial properties. The incorporation of polyphosphazenes of formula I as a component of the disclosed MFWDM of interest can significantly reduce bacterial attachment to the MFWDM, and thereby, promote the maintenance of a sterile environment. In various embodiments, the disclosed MFWDM incorporating polymers of formula I can exhibit odor-adsorbing properties, as a result of preventing bacterial infiltration.

In various embodiments, the disclosed multi-functional wound-care dressing matrices incorporating polyphosphazenes of formula I as a component, can exhibit lubricious, or non-stick, non-adherent, and liquid-repellent properties. The incorporation of polyphosphazenes of formula I as a component of the disclosed MFWDM of interest can significantly reduce the degree of attachment between the tissue-contacting surface of a MFWDM and the delicate epithelial layer of a given wound site. Detachment of MFWDM from the surface of biological substrates, such as various cellular tissues of human and animal subjects, without incurring additional tissue injury to the biological substrate provides significant advantages by reducing secondary complications introduced during the removal process, such as prematurely re-opening unhealed wounds, rupturing the integrity of surrounding tissue, or increasing the risk of inviting pathogenic infections. Thus, the non-stick, moisture-repellent properties of polyphosphazenes of formula I can promote tissue healing and enable the removal of a MFWDM with minimal discomfort and pain.

In various embodiments, the disclosed multi-functional wound-care dressing matrices incorporating polyphosphazenes of formula I as a component, can exhibit fluid-repelling, fluid-adhering (wetting) or fluid-transporting properties, the latter properties being not exclusively a function of surface energy and density of the MFWDM material. The incorporation of polyphosphazenes of formula I as a component of the disclosed MFWDM of interest can therefore act to stabilize the desired interfacial properties of the device when being in contact with a fluid or facilitate transport of the fluid through the protective barrier. In one embodiment of the aforementioned MFWDM material, a fluid-repellency property can act to maintain a liquid substantially above the protective barrier material contacting the wound, or below the materials' surface facing the wound. Thus, this effect may assist in helping to contain for instance contagious, infectious or septic fluids below the protective barrier materials' surface (facing the wound), increasing the medical safety of the personnel being in direct contact with the (wounded) person. Another potentially desired effect of this embodiment is shielding the wound from liquids or moisture penetrating through the protective carrier material, which will also help in maintaining the devices durability, (adhesiveness) and the desired environment/moisture state of the wound. Additionally it can prevent the emergence of bad-odors (such as arising from the breakdown of organic material, bacteria, necrotic tissue at the site of the wound) to the external environment, adding additional comfort to patient and health care personnel. In another embodiment of the aforementioned MFWDM material, liquid wetting as a feature can help to maintain a certain degree of liquid saturation within the wound or actively promote the transport of liquids (e.g. containing pharmaceutical agents) into the wound, e.g., for wound moisturization (e.g. preceding the planned removal of the protective barrier) or ease medical treatment. The balance of fluid-repelling or adhering properties can express itself in terms of fluid-transporting ability.

In various embodiments, the disclosed multi-functional wound-care dressing matrices incorporating polyphosphazenes of formula I as a component, can exhibit outstanding biostability properties by not reacting with components of physiological fluids over prolonged period of time. The incorporation of polyphosphazenes of formula I as a component of the disclosed MFWDM can impart exceptional bio-inertness in order to provide a passive barrier that can function as an effective protective physical barrier, as well as a moisture barrier. In various embodiments, the disclosed multi-functional wound-care dressing matrices incorporating polyphosphazenes of formula I as a component, can exhibit outstanding biostability properties by not reacting with components of physiological fluids over prolonged period of time. The incorporation of polyphosphazenes of formula I as a component of the disclosed MFWDM can impart exceptional bio-inertness in order to provide a passive barrier that functions as an effective protective barrier. As a secondary benefit of the stability in physiological or other fluids, a MFWDM can serve as an intermediate layer mediating between e.g., a liquid or gel based ointment or reservoir for additional agent transport from the reservoir to the wound environment.

3. Definition of Polymers of Formula I

In various embodiments, multi-functional wound-care dressing matrices of the present disclosure comprises polymers of general formula (I). Various embodiments are directed to multi-functional wound-care dressing matrices comprising a polymeric compound poly[bis(trifluoroethoxy) polyphosphazene] and/or derivatives thereof.

Various embodiments are directed to multi-functional wound-care dressing matrices (“MFWDM”) comprising a substrate layer formed as a wound dressing matrix comprising at least one tissue-contacting surface that incorporates at least one polymer component having the general formula (I):

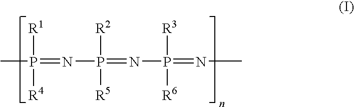

in which the n value is an integer from 2 to ∞;

R¹ to R⁶ are independently selected from:
a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
a heterocyclic alkyl group with at least one nitrogen, phosphorus, oxygen, sulfur, or selenium as a heteroatom;
a heteroaryl group with at least one nitrogen, phosphorus, oxygen, sulfur, or selenium as the heteroatom;
a nucleotide or a nucleotide residue;
a biomacromolecule; or
a pyrimidine or a purine base.

Suitable substituents for R¹ to R⁶ can be independently selected from: halide substituents, such as fluorine, chlorine bromine, or iodine; pseudohalide substituents, such as cyano (—CN), isocyano (—NC), thiocyano (—SCN), isothiocyano (—NCS), cyanato (—OCN), isocyanato (—NCO), azido (—N₃) groups; substituents such as nitro-(—NO₂) and nitrito (—NO) groups; partially substituted alkyl groups, such as haloalkyl; heteroaryl such as imidazoyl, oxazolyl, thiazolyl, pyrazolyl derivatives; or purine and pyrimidine bases such as guanidines, amidines and other ureido derivatives of the base structure.

As used herein, alkyl (R), alkoxy (—OR), alkylsulfonyl (—SO2R), alkyl amino (—NHR), dialkyl amino (—NR2), carboxylic acid ester (-(alkadiyl)C(O)OR or -alkadiyl)OC(O)R)), ureido (—NHC(O)NH2, —NRC(O)NH2, —NHC(O)NHR, —NRC(O)NHR, —NHC(O)NR2, —NRC(O)NR2, and their alkadiyl-linked analogs), alkylmonoamidine (including —N═C(NR2)R, -(alkadiyl)N═C(NR2)R, —C(NR2)═NR, and -(alkadiyl)C(NR2)═NR), alkylbisamidine (including —N═C(NR2)2, -(alkadiyl)N═C(NR2)2, —NRC(NR2)═NR, and -(alkadiyl)NRC(NR2)═NR), alkoxymonoamidine (—O(alkadiyl)N═C(NR2)R, —OC(NR2)═NR, and —O(alkadiyl)C(NR2)═NR)), and alkoxybisamidine (—O(alkadiyl)N═C(NR2)2. —O(alkadiyl)NRC(NR2)═NR, and O(alkadiyl)NRC(NR2)═NR) moieties are defined by the corresponding formula shown, in which R can be selected independently from a linear, branched, and/or cyclic (“cycloalkyl”) hydrocarbyl moieties, including alkyl (saturated hydrocarbons) as well as alkenyl and alkynyl moieties, having from 1 to 20 (for example, from 1 to 12, or 1 to 6) carbon atoms.

The inclusion of alkenyl and alkynyl moieties provides, among other things, the capability to cross-link the polyphosphazene moieties to any extent desired. Examples of alkyl groups include, but are not limited to, methyl, ethyl, propyl, isopropyl, n-butyl, t-butyl, isobutyl, sec-butyl, pentyl, isopentyl, neopentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl).

According to this definition and usage (supra), specific examples of R (alkyl) groups include unsubstituted alkyl, substituted alkyl such as halo-substituted alkyl (haloalkyl), unsubstituted alkenyl, substituted alkenyl such as halo-substituted alkenyl, and unsubstituted alkynyl, and substituted alkynyl such as halo-substituted alkynyl.

Furthermore, these examples of R (alkyl) provide that the alkoxy (OR) substituents can be unsubstituted alkoxy (“alkyloxy”), substituted alkoxy such as halo-substituted alkoxy (haloalkoxy), unsubstituted alkenyloxy, substituted alkenyloxy such as halo-substituted alkenyloxy, unsubstituted alkynyloxy, and substituted alkynyloxy such as halo-substituted alkynyloxy. In this aspect, vinyloxy and allyloxy can be useful.

A silyl group is a —SiR3 group and a silyloxy group is an —OSiR3 group, where each R moiety is selected independently from the R groups defined supra. That is, R in each occurrence is selected independently from a linear, branched, and/or cyclic (“cycloalkyl”) hydrocarbyl moieties, including alkyl (saturated hydrocarbons) as well as alkenyl and alkynyl moieties, having from 1 to 20 (for example, from 1 to 12, or 1 to 6) carbon atoms.

Unless otherwise specified, any R group can be unsubstituted or substituted independently with at least one substituent selected from a halogen (fluorine, chlorine, bromine, or iodine), an alkyl, an alkylsulfonyl, an amino, an alkylamino, a dialkylamino, an amidino (—N═C(NH2)2), an alkoxide, or an aryloxide, any of which can have up to 6 carbon atoms, if applicable. Thus, the term substituted “alkyl” and moieties which encompass substituted alkyl, such as “alkoxy”, include haloalkyl and haloalkoxy, respectively, including any fluorine-, chlorine-, bromine-, and iodine-substituted alkyl and alkoxy. Thus, terms haloalkyl and haloalkoxy refers to alkyl and alkoxy groups substituted with one or more halogen atoms, namely fluorine, chlorine, bromine, or iodine, including any combination thereof.

Unless otherwise indicated, the term “aryl” means an aromatic ring or an aromatic or partially aromatic ring system composed of carbon and hydrogen atoms, which may be a single ring moiety, or may contain multiple rings bound or fused together. Examples of aryl moieties include, but are not limited to, phenyl, anthracenyl, azulenyl, biphenyl, fluorenyl, indan, indenyl, naphthyl, phenanthrenyl, 1,2,3,4-tetrahydro-naphthalene, tolyl, and the like, any of which having up to 20 carbon atoms. An aryloxy group refers to an —O(aryl) moiety.

The terms haloaryl and haloaryloxy refer to aryl and aryloxy groups, respectively, substituted with one or more halogen atoms, namely fluorine, chlorine, bromine, or iodine, including any combination thereof.

A heterocyclic alkyl group with at least one nitrogen as a heteroatom refers to a non-aromatic heterocycle and includes a cycloalkyl or a cycloalkenyl moiety in which one or more of the atoms in the ring structure is nitrogen rather than carbon, and which may be monocyclic or multicyclic, and may include exo-carbonyl moieties and the like. Examples of heterocyclic alkyl group with nitrogen as a heteroatom include, but are not limited to, piperazinyl, piperidinyl, pyrrolidinyl, tetrahydropyrimidinyl, morpholinyl, aziridinyl, imidazolidinyl, 1-pyrroline, 2-pyrroline, or 3-pyrroline, pyrrolidinonyl, piperazinonyl, hydantoinyl, piperidin-2-one, pyrrolidin-2-one, azetidin-2-one, and the like. Thus, these groups include heterocyclic exocyclic ketones as well.

A heteroaryl group with at least one nitrogen as the heteroatom refers to an aryl moiety in which one or more of the atoms in the ring structure is nitrogen rather than carbon, and which may be monocyclic or multicyclic. Examples of heterocyclic alkyl group with nitrogen as a heteroatom include, but are not limited to, acridinyl, benzimidazolyl, quinazolinyl, benzoquinazolinyl, imidazolyl, indolyl, isothiazolyl, isoxazolyl, oxazolyl or oxadiazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, triazinyl, and the like. In this aspect, this disclosure includes or encompasses chemical moieties found as subunits in a wide range of pharmaceutical agents, natural moieties, natural biomolecules, and biomacromolecules. For example, this disclosure encompasses a number of pharmaceutical agents available with the tetrazole group (for example, losartan, candesartan, irbesartan, and other Angiotensin receptor antagonists); the triazole group (for example, fluconazole, isavuconazole, itraconazole, voriconazole, pramiconazole, posaconazole, and other antifungal agents); diazoles (for example, fungicides such as Miconazole, Ketoconazole, Clotrimazole, Econazole, Bifonazole, Butoconazole, Fenticonazole, Isoconazole, Oxiconazole, Sertaconazole, Sulconazole, Tioconazole, and the like); and imidazoles (histidine, histamine, and the like). Thus in one aspect, some of the R1 to R6 moieties in the formula I can encompass chemical moieties found as subunits in a wide range of pharmaceutical agents, natural moieties, natural biomolecules, and biomacromolecules.

A heterocyclic alkyl group with at least one phosphorus, oxygen, sulfur, or selenium as a heteroatom refers to a non-aromatic heterocycle and includes a cycloalkyl or a cycloalkenyl moiety in which one or more of the atoms in the ring structure is phosphorus, oxygen, sulfur, or selenium rather than carbon, and which may be monocyclic or multicyclic, and may include exo-carbonyl moieties and the like. Similarly, a heteroaryl group with at least one phosphorus, oxygen, sulfur, or selenium as the heteroatom refers to an aryl moiety in which one or more of the atoms in the ring structure is phosphorus, oxygen, sulfur, or selenium rather than carbon, and which may be monocyclic or multicyclic. Examples of heterocyclic alkyl groups or heteroaryls with phosphorus, oxygen, sulfur, or selenium as a heteroatom include, but are not limited to, substituted or unsubstituted ethylene oxide (epoxides, oxiranes), oxirene, oxetane, tetrahydrofuran (oxolane), dihydrofuran, furan, pyran, tetrahydropyran, dioxane, dioxin, thiirane (episulfides), thietane, tetrahydrothiophene (thiolane) dihydrothiophene, thiophene, thiane, thiine (thiapyrane), oxazine, thiazine, dithiane, dithietane, and the like. Thus, these groups include all isomers, including regioisomers of the recited compounds. For example, these groups include 1,2- and 1,3-oxazoles, thiazoles, selenazoles, phosphazoles, and the like, which include different heteroatoms from the group 15 or group 16 elements.

4. Exemplary Methods for Forming a Stable Coating Layer of Polymers of Formula I onto Substrates of Interest

As an exemplary method, a stable bonding can be formed between a substrate of interest and a coating layer comprising polymers of formula I by introducing chemical modifications at the interface between the substrate surface and the coating layer. A suitable interface can be introduced by inducing the formation of copolymers, e.g., random copolymers, alternating copolymers, block copolymers, graft copolymers, blends, or interpenetrating networks between a polymer substrate surface of interest and polymers of formula I.

For example, ‘A’ refers to the backbone of a polymer of formula I, ‘B’ refers to the backbone of a polymer of the substrate surface. The following illustrations shall help understand the concept:

Side groups are omitted in this depiction.

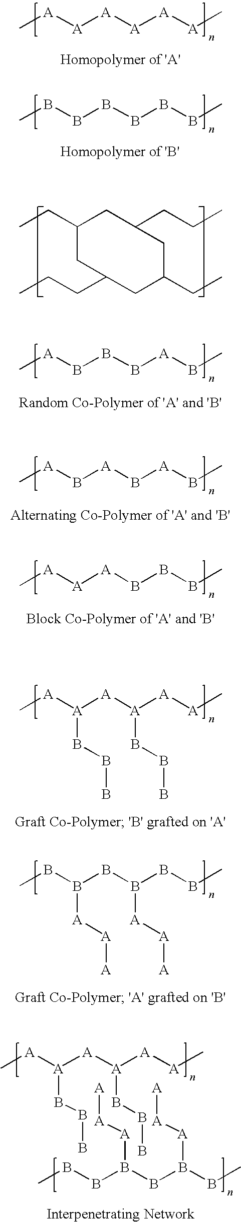

Other than the connectivities described in this illustration, the connectivities can be not only achieved by connecting backbone to backbone units as depicted, but it may also include one or more side group(s) of one polymer connecting to one or more backbone units of the other polymer, or connections of one or more side group(s) of one polymer to one or more side group(s) of the other polymer, and all possible permutations thereof. Furthermore, these connectivities are not limited to two polymers forming a copolymer, but it also may include a third or more polymers, or a suitable linking moiety participating in the bond formation between backbone or side group units. This definition therefore also encompasses tie layers composed of ethyleneimines or aminosilanes, and the like as described for coating.

A blend of polymers can be described as any arbitrary mixture of polymer ‘A’ in ‘B’, commonly formed by using a suitable cosolvent for each polymer, or using a melt. A formation of a homogeneous or intergradient blend is preferred over the formation of a heterogeneous blend with more than one phase.

An interpenetrating network can be understood of polymer chains (backbone units with side groups) diffusing from one polymer into the other and interacting with polymer chains of the other in order to create a proper adhesion between the different polymers. In the context of this invention, the term semi-interpenetrating network is preferred, as one polymer (the base substrate) may consist of crosslinked polymer chains, while the other (top-) polymer (polymers of formula I) may be non-crosslinked and is diffusing into the other polymer. A semi-interpenetrating network differs from the interpenetrating network by one or more polymer(s) being crosslinked and forming a stable network matrix while the other polymer is non-crosslinked. In a true interpenetrating network both polymers may be crosslinked. Copolymer formation techniques are provided below:

Several strategies are valid to bring about formation of any of the above described copolymers. Copolymers may be formed by “co”-polymerizing a suitable mixture of precursors (monomer units or very small, low molecular weight molecule units) of both polymers at the same time. Depending on the conditions (simultaneous or stepwise reaction, self-organizing/assembling reaction . . . ) used, this can provide examples for forming random, alternating, block copolymers, blends, or (semi)-interpenetrating network of both or more polymers all together.

‘A’ grafted on ‘B’

By attaching these monomer/precursor units of one polymer to the other polymer and then subsequently polymerizing these monomer units while being ‘grafted’ on the backbone of the other polymer, a stable copolymer can be formed. In this context, this could mean co-polymerizing suitable phosphazene precursors with suitable precursors or polymer chains from the base substrate. This is an example of the method ‘A’ grafted on ‘B’, where chains of polymers of formula I (and/or their precursors) are grafted on the backbone of the base substrate polymer. This type of grafting process may also involve a stepwise increase in molecular weight of the grafted side chains of polymers of formula I in relation to the distance of the pure base substrate polymer phase to the pure polymers of formula I phase. A gradual shift in molecular weight will increase the diffusion of the polymers of formula I into the base substrate polymer phase while allowing a gradual transition in surface energy, reducing the risk of phase separation or adhesive failure. Suitable precursors for polymers of formula I are composed as follows:

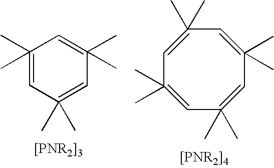

Cyclic Phosphazene Precursors Used During Ring Opening Polymerization

The pendant groups R— can be composed of halogen elements, such as Fluorine, Chlorine, Bromine, and Iodine atoms. Within this scenario most preferentially used is Chlorine, for which there exists prior known art. Furthermore, the group R— can encompass any known analogues of main group VII elements, i.e. isolobal (isoelectronic) fragments. Exemplary isolobal fragments can include, but are not limited to cyano, thiocyanate, cyanate, and azide groups. Other common organic side groups such as, —COOH, —NH₂ can also be used as suitable pendant side groups, provided that the electronegative substituent character allows for a similar plasma reactivity as demonstrated for Chlorine substituents. In the most preferred embodiment the pendant side groups R— are composed of ether moieties —OR, such as —OEt, —OMet, but most preferably —OCH₂CF₍₃₋ₘ₎, where m=0-2. Also, the suitable phosphazene precursors might not only be cyclic but include linear, lower molecular weight polymers of formula I or crosslinked chains of polymers of formula I. This type of grafting could also be achieved by using polymers of formula I that contain base substrate anchor groups in end positions of the polymer.

‘B’ grafted on ‘A’

In another case, the co-polymer may be formed by grafting reactive base substrate groups to the polymers of formula I backbone with suitable, reactive short chain side groups. Other strategies in copolymer formation include the linking of side groups by suitable reagents.

Interpenetrating Network (IPN)

The success of forming an interpenetrating network will mainly depend on creating a stable, homogeneous mixture of the two polymers that is mediated by a suitable cosolvent that will have the right degree of solubility of one polymer while maintaining enough solubility for the other polymer, so both polymer phases do not separate. The formation of a stable interpenetrating network may involve a stepwise deposition of polymers of formula I layers with increasing molecular weight of the deposited polymers of formula I in relation to the distance of the pure base substrate polymer phase to the pure, high molecular weight polymers of formula I phase. A gradual shift in molecular weight will increase the diffusion of the polymers of formula I into the base substrate polymer phase while allowing a gradual transition in surface energy, reducing the risk of unwanted phase separation or adhesive failure. Also, the initial bonding of a primary layer of polymers of formula I to a base substrate may involve deposition of suitable precursors as described previously, with a subsequent thermal, radiation-induced, or plasma-induced polymerization, crosslinking reaction of the polymers of formula I or precursors thereof described previously interdiffused within the base substrate domain. Importantly, for any of above mixtures described, a phase separation during the curing phase of the polymeric mixture may be desired. Due to the mostly hydrophobic nature of the polymers of formula I, there will be a trend for the hydrophobic part of the mixture to be located or concentrated towards the outside of a micellic structure (due to surface energy) during curing as the monomer part that will form the base polymer substrate of the finished part, will be depleted during curing.

### C. Exemplary Multi-Functional Wound-Care Dressings Incorporating Polymers of Formula I

As described above, the multi-functional wound-care dressing matrices (“MFWDM”) comprising polymers of formula I and various polymeric networks of interest can be made to have an open, or closed, or semi-closed cell-design. The porosity of these structures can further be nano-, meso-, micro-, or macro-porous. The structure can further be composed of cellular, fibrous, fibrillar, porous or capillary, or cylindrical or tubular elements, all of which may be arranged in an isotropic, anisotropic, or symmetric respectively asymmetric fashion, or can contain a gradient in terms of having elements of de- or increasing sizes, or structures thereof.

Alternatively, the multi-functional wound-care dressing matrices MFWDM comprising the polymers of formula I can be created as closed, partially closed or open, porous, or semi-porous, smooth or rough, or specifically structured or textured films and layers. These films or layers, and their respective structural elements can be created in dimensions ranging from nanometers over micrometers to millimeters. As a logical extension, the repetition, combination or multiplication of such structural and dimensional parameters allows the extension of the presented size ranges to larger scales.

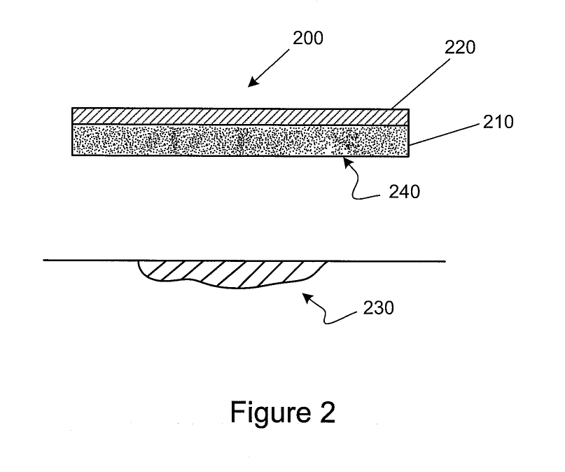

FIG. 2 is a schematic of a multi-functional wound-care dressing matrix comprising multiple substrate layers, as one embodiment of the present disclosure. In FIG. 2, the MFWDM 200 comprises two layers of substrate layers 210 and 220 juxtaposed together. The polymers of formula I can be incorporated into either or both membranes 210 and 220. The two layers of substrate layers can be juxtaposed in any manner, including adhesive, blending, dip-coating, spray-coating,______. The substrate layers are suitable as films, membranes, meshes, foils, gauzes, pads, foams, sponges, or equivalents of any dimension or shape, known to persons skilled in the art. When positioned over a wound site 230, the tissue-contacting surface 240 of the MFWDM makes direct, or indirect, contact with the bodily fluids, such as blood or exudate, and/or cellular tissue matter. The number of layers of substrate layers composing a MFWDM can range from about 2 to about 30, from about 2 to 25, from about 2 to 20, from about 2 to 15, from about 2 to 10, and from about 2 to 5. The description of a substrate layer in FIG. 1 applies to embodiments described in FIGS. 2-4.

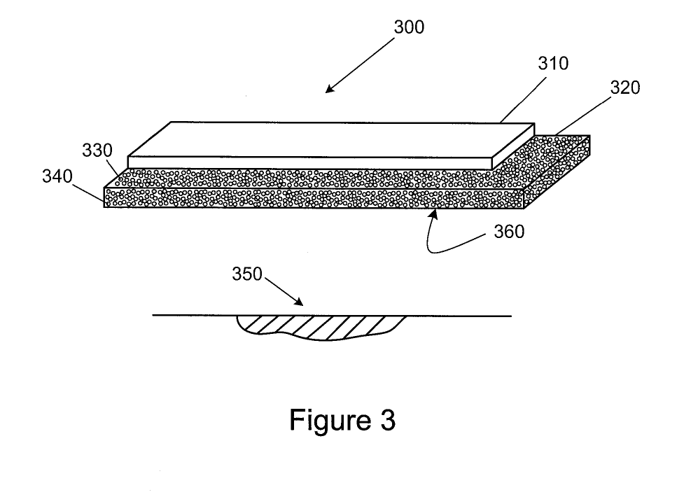

FIG. 3 is a schematic of a multi-functional wound-care dressing matrix comprising multiple layers, and includes capsules, as one embodiment of the present disclosure. In FIG. 3, the MFWDM 300 comprises two layers of substrate layers 310 and 320 juxtaposed together in any manner. The MFWDM 300 further comprises capsules, such as 330 and 340, comprising one or more agents of interest, that can represent a mixture of agents of interest selected from a multitude of drugs, bioactive agents, or other compounds or compositions of interest, natural or synthetic, known to persons skilled in the art, that can promote healing and stimulate new tissue growth. The capsules can be composed of natural or synthetic, biodegradable polymer, or a blend thereof. When positioned over a wound site 350, the tissue-contacting surface 360 of the MFWDM makes direct, or indirect, contact with the bodily fluids, such as blood or exudate, and/or cellular tissue matter. The number of layers of substrate layers composing a MFWDM can range from about 2 to about 30, from about 2 to 25, from about 2 to 20, from about 2 to 15, from about 2 to 10, and from about 2 to 5.

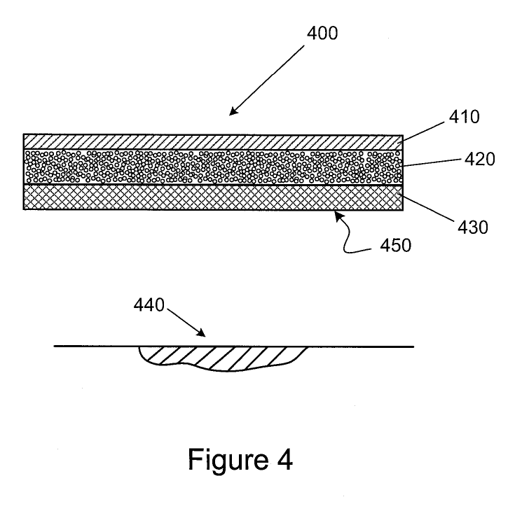

FIG. 4 is a schematic of a multi-functional wound-care dressing matrix comprising multiple substrate layers, and includes polymers of formula I formulated as a foam/sponge, as one embodiment of the present disclosure. In FIG. 4, the MFWDM 400 comprises at least three layers of substrate layers: a permeable or non-permeable polyphosphazene layer 410, situated above a hydrogel reservoir suitable for liquid uptake 420, situated above a permeable polyphosphazene layer 430, in which the substrate layers can be juxtaposed together in any manner, and can be produced by electrospinning, spray-coating, or other established methods known to persons skilled in the art. The permeable polyphosphazene layer 430 can be produced to exhibit porous, fibrous, or capillary substructures, including forms such as foams, sponges, films, woven or non-woven membranes, or equivalents known to persons skilled in the art. When positioned over a wound site 440, the tissue-contacting surface 450 of the MFWDM makes direct, or indirect, contact with the bodily fluids, such as blood or exudate, and/or cellular tissue matter. The number of layers of substrate layers composing a MFWDM can range from about 2 to about 30, from about 2 to 25, from about 2 to 20, from about 2 to 15, from about 2 to 10, and from about 2 to 5.

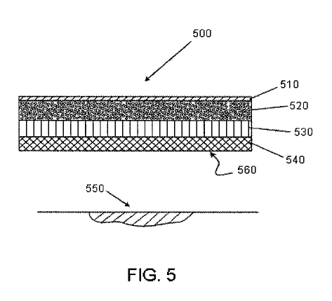

FIG. 5 is a schematic of a multi-functional wound-care dressing matrix comprising multiple substrate layers, and formulated as a adhesive wound patch, as one embodiment of the present disclosure. In FIG. 5, the MFWDM 600 comprises at least four layers of substrate layers: a polyphosphazene-derived top layer 510, situated above a liquid absorbent hydrogel layer (as a form/sponge for example) 520, situated above a permeable polyphosphazene layer 530, situated above an adhesive layer 540, in which the substrate layers can be juxtaposed together in any manner, and can be produced by electrospinning, spray-coating, or other established methods known to persons skilled in the art. The hydrogel layer 520 can be composed of carylate, hyaluronate, alginate, chitosane, polyethylene oxide or PHEMA polymer derivates. The adhesive layer 540 can be composed of biodegradable polymer, or cyanoacrylate, or cellulose acetate or polyurethane, and can be made to be activated by light, heat, or moisture. When positioned over a wound site 550, the tissue-contacting surface 560 of the MFWDM makes direct, or indirect, contact with the bodily fluids, such as blood or exudate, and/or cellular tissue matter. The number of layers of substrate layers composing a MFWDM can range from about 2 to about 30, from about 2 to 25, from about 2 to 20, from about 2 to 15, from about 2 to 10, and from about 2 to 5.

In various embodiments, including those described in FIGS. 1-5, the MFDWM incorporating polymers of formula I further comprises a tubing member that can attach to the MFDWM in a manner that permits the withdrawal of bodily fluids when the tissue-contacting surface of the MFDWM is positioned over a wound site. In various embodiments, the substrate layer comprising the tissue-contacting surface is one or more foam/sponges that can be fused together with other very absorbent, porous, and durable materials. The excess exudate can be removed from the wound site by at least the capillary structure of the tissue-contacting layer comprising polyphosphazenes of formula I when a negative pressure (a vacuum) is applied employing an external source, which can be a manually operated or an automated vacuum-producing pump or equivalents thereof.

The present disclosure relates to the technology to prepare and apply tailor-made polyphosphazene matrices in form of 3-dimensional bulk (volume) materials and/or 2-dimensional films of arbitrary shape and form (such as films, fibers, membranes, slabs, sponges, foams, pads, spherical, cylindrical, layered, compositions), composite or pure material (augmenting or constituting entirely an underlying structure of a device or being composed of several components), that convey improved beneficial properties to the targeted application, the desired function of a device, or the device itself by being able to control specific polyphosphazene matrix properties such as porosity, permeation, diffusion, structural and dimensional range (such as film thickness, and lateral dimensions), elastic modulus, refractive index, surface energy, cohesive energy density as well as surface or bulk morphology. Further, the polyphosphazene matrices being targeted for topological appliances in wound care medicine, having the purpose of serving as a protective barrier material or an otherwise desired function.

The aforementioned physical properties of the polyphosphazene matrix materials can be shown to exert a direct influence on biomedical characteristics when employed as a biomaterial. Some of the aforementioned properties of a biomaterial include, for example, cellular and bacterial adhesion or proliferation thereof, tendency of organic or inorganic encrustation and matter build-up, activation of the blood coagulation cascade, the risk of thrombosis formation or the activation of the complement system and its effect on biological acceptance and blending in of a medical device with a host subject. Other important properties for the device serving as a protective barrier material include gas and liquid permeation, transport or diffusion, such as air and aqueous fluids, blood, serum, inter- and intracellular fluids, pharmaceutical agents, resistance to bacterial infiltration and generally the ability to protect from environmental conditions, such as moisture, temperature conditions (heat/cold), mechanical impacting, abrasion and the like.

The ability to control the physical properties of the polyphosphazene matrix materials constitutes a major improvement in the development of medical protective barrier/wound care devices and (ways by which) polyphosphazene matrix materials (can be used) useful for medical devices and applications. Hence, the desired field of application for this technology and the major emphasis and range of applications are focused on modern medical implant technology and the deployment of intelligent biomaterials. This is not meant to limit the range of the presented technology or its potential applications and further specific examples will be given.

Plasticizers, lubricating agents, adhesives, polymer additives in general as well as polymeric breakdown products may surface migrate and leach over time from the device, thereby not only causing a detrimental alteration of the mechanoelastical properties, but also affecting biological properties and potentially causing an undesired biological response of the protective barrier device over the course of deployment time, thereby gradually lessening or destroying the biological compatibility of the device. The Polyzene®-F solutions, employed in the Example formulations, provided below, can be mixed with other polymeric agents, adhesives, adhesion promoters, blowing agents, filling agents, pharmaceutical agents in order to afford an accordingly blended membrane material.

Various embodiments are directed to methods for producing multi-functional wound-care dressing matrix, comprising:

incorporating onto at least one tissue-contacting surface of a substrate layer formed as a wound dressing,

at least one high molecular weight polyphosphazene polymer of formula (I):

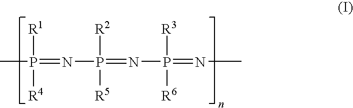

wherein

n is an integer from about 40 to about 100,000;

R¹ to R⁶ are independently selected from:
a) a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
b) a heterocyclic alkyl group with at least one nitrogen, phosphorus, oxygen, sulfur, or selenium as a heteroatom;
c) a heteroaryl group with at least one nitrogen, phosphorus, oxygen, sulfur, or selenium as the heteroatom;
d) a nucleotide or a nucleotide residue;
e) a biomacromolecule; or
f) a pyrimidine or a purine base.

Various embodiments are directed to methods for healing wounds, the method comprising:

covering a wound site with a multi-functional wound-care dressing matrix, comprising:

a substrate layer formed as a wound dressing comprising at least one tissue-contacting surface incorporating

at least one high molecular weight polyphosphazene polymer of formula (I):

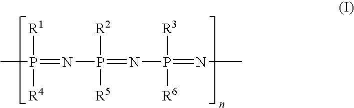

wherein

n is an integer from about 40 to about 100,000;

R¹ to R⁶ are independently selected from:
a) a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
b) a heterocyclic alkyl group with at least one nitrogen, phosphorus, oxygen, sulfur, or selenium as a heteroatom;
c) a heteroaryl group with at least one nitrogen, phosphorus, oxygen, sulfur, or selenium as the heteroatom;
d) a nucleotide or a nucleotide residue;
e) a biomacromolecule; or
f) a pyrimidine or a purine base; and

permitting sufficient time for the wound to heal.

All publications and patents mentioned in this disclosure are incorporated herein by reference in their entireties, for the purpose of describing and disclosing, for example, the constructs and methodologies that are described in the publications, which might be used in connection with the presently described methods, compositions, articles, and processes. The publications discussed throughout the text are provided solely for their disclosure prior to the filing date of the present application. Nothing herein is to be construed as an admission that the inventors are not entitled to antedate such disclosure by virtue of prior invention. Should the usage or terminology used in any reference that is incorporated by reference conflict with the usage or terminology used in this disclosure, the usage and terminology of this disclosure controls. The Abstract of the disclosure is provided to satisfy the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” The Abstract is not intended to be used to construe the scope of the appended claims or to limit the scope of the subject matter disclosed herein. Moreover, any headings are not intended to be used to construe the scope of the appended claims or to limit the scope of the subject matter disclosed herein. Any use of the past tense to describe an example otherwise indicated as constructive or prophetic is not intended to reflect that the constructive or prophetic example has actually been carried out.

Also unless indicated otherwise, when a range of any type is disclosed or claimed, for example a range of molecular weights, layer thicknesses, concentrations, temperatures, and the like, it is intended to disclose or claim individually each possible number that such a range could reasonably encompass, including any sub-ranges encompassed therein. For example, when the Applicants disclose or claim a chemical moiety having a certain number of atoms, for example carbon atoms, Applicants' intent is to disclose or claim individually every possible number that such a range could encompass, consistent with the disclosure herein. Thus, by the disclosure that an alkyl substituent or group can have from 1 to 20 carbon atoms, Applicants intent is to recite that the alkyl group have 1, 2, 3, 4, 5, 6, 7, 8, 9, 10, 11, 12, 13, 14, 15, 16, 17, 18, 19, or 20 carbon atoms, including any range or sub-range encompassed therein. Accordingly, Applicants reserve the right to proviso out or exclude any individual members of such a group, including any sub-ranges or combinations of sub-ranges within the group, that can be claimed according to a range or in any similar manner, if for any reason Applicants choose to claim less than the full measure of the disclosure, for example, to account for a reference that Applicants are unaware of at the time of the filing of the application.

### EXAMPLES

### Example 1

### Preparation of Thin Polyzene®-F Matrix by Dip-Coating Method

In one embodiment, thin films of Polyzene®-F Matrix can be prepared by dip-coating method, as follows. To produce ultra-thin Polyzene®-F films on arbitrary substrates, a programmable dip-coating stage can be employed. Pre-cleaned and adhesion promoter pre-treated substrates can be immersed into solutions of Polyzene®-F in concentration ranges from 0.5 to 50 mg/ml in various solvents for a period of 0-5 min., after which they can be withdrawn from the solution at a speed of 50 μm/s up to 50 mm/min. After removal of the samples, an optional heat curing step at 40-80° C. for 10-30 min can be employed to achieve removal of residual solvents. The resulting films exhibit thickness from a range from about 0 to about 1.0 μm, and from about 0-0.5 μm.

### Example 2

### Preparation of Thin Polyzene®-F Matrix by Spin-Coating Method

In one embodiment, thin films of Polyzene®-F Matrix can be prepared by spin-coating method, as follows. To produce homogeneous and ultrathin Polyzene®-F films on flat substrates, a spin-coating procedure can be employed. The pre-cleaned and adhesion promoter pre-treated substrates can be centered on a spin-coating device and 0.1 to 0.5 ml of Polyzene®-F solutions in concentration ranges from 0.5 to 50 mg/ml in various solvents are spread on the substrate. After a dwell time of 1-10 sec, a ramp to 10-1000 rpm can be executed to achieve homogeneous spreading of the solution, followed by a linear ramp to a target of 1000-2000 rpm for further thinning of the film in an interval time of 1-10 seconds. A final ramp to 2000-4000 rpm with a dwell time of 5-120 sec can be carried out to arrive at the desired film thickness. After removal of the samples, an optional heat curing step at 40-80° C. for 10-30 min. can be employed to achieve removal of residual solvents. The resulting films exhibit thickness from a range from about 0-0.5 μm.

### Example 3

### Preparation of Thick Polyzene®-F Matrix by Spray-Coating Method

In one embodiment, thin films of Polyzene®-F Matrix can be prepared by a spray-coating method, as follows. A pneumatic dual-feed coaxial nozzle with an orifice of 0.5 mm can be supplied with Polyzene®-F solutions in various solvent blends using a programmable syringe pump. Polyzene®-F concentration ranges from 0 to 20 mg/ml, and can be supplied to the nozzle at a rate of 1-5 ml/min. Atomization can be achieved by pressure regimes of 1.0-4.5 bar depending on the viscosity of the solution. Sample distance can be varied in the experiment between 0-40 cm. The resulting films exhibit a thickness from a range from about 1.0 to about 100 μm, depending on the employed spray-coating time period.

### Example 4

### Preparation of Polyzene®-F Membranes by Phase-Separation Method—Thermogelation of a Homogeneous Solution of Two or More Components

In one embodiment, Polyzene®-F membranes can be prepared by phase separation method, as follows. Polyzene®-F and a suitable solvent capable of forming a homogeneous solution at elevated temperature, which upon cooling exhibits a miscibility gap, and can lead to the precipitation of the polymer. Examples of suitable combinations include Polyzene®-F and Ethylene glycol dimethyl ether, t-Butyl methyl ether, Ethyl octanoate or Cyclohexanone. A gel-like Polyzene®-F layer can be formed during cooling of supersaturated Polyzene®-F solutions with these solvents.

Exemplary Formulations: a) 10-100 mg Polyzene®-F can be dissolved in 1-10 ml t-Butylmethyl ether under reflux (boiling point) conditions; and b) 10-100 mg Polyzene®-F dissolved in 1-10 ml Ethyl octanoate at 80° C. A slightly opaque, gel-like Polyzene®-F layer can be formed during cooling of the saturated Polyzene®-F solution to ambient temperature. The gel-layer can further be obtained as a highly porous solid by e.g., gradual solvent exchange with non-solvent (using a cryoextraction procedure) or by supercritical point drying (state of the art technique). The resulting membranes exhibit thickness from a range from about 0.1 to about 100 μm.

### Example 5

### Preparation of Polyzene®-F Membranes by Phase-Separation Method—Evaporation of a Volatile Solvent from a Homogeneous Solution of Two or More Components

In one embodiment, Polyzene®-F membranes can be prepared by phase separation method, as follows. Controlled evaporation of a volatile solvent from a two (solvent/Polyzene®-F) or three component (solvent/precipitant/Polyzene®-F) homogeneous blend, can lead to the precipitation of a polymer enriched phase. In a three component mixture, the precipitant to be chosen is usually a less volatile nonsolvent. Precipitation can be induced by evaporation of the volatile solvent as the solvent mixture gradually becomes enriched with precipitant. Examples of suitable combinations include Polyzene®-F and Acetone, THF or Ethyl acetate for typical solvent cast films. Depending on evaporation rate and Polyzene®-F concentration, the examples above can give slightly porous up to completely closed, transparent, spherulitic Polyzene®-F films. Suitable three component mixtures include Polyzene®-F and Acetone/Isopropanol or Ethyl acetate/Isopropanol blends or any other suitable solvent/nonsolvent mixture. Membranes prepared by this method can form opaque films with porous to fibrous character. The resulting membranes exhibit thickness from a range from about 0.1 to about 100 μm.

### Example 6

### Preparation of Polyzene®-F Membranes by Phase-Separation Method—Evaporation of a Volatile Solvent from a Homogeneous Solution of Two or More Components

In one embodiment, Polyzene®-F membranes can be prepared by phase separation method, as follows. Two-component mixtures tested (typical solvent cast films): Polyzene®-F/Acetone; Polyzene®-F/THF; and Polyzene®-F/Ethyl acetate. Concentration ranges tested is 0.5-2 (w/v) % Polyzene®-F/Solvent. Depending on evaporation rate and Polyzene®-F concentration, the examples above can give slightly porous up to completely closed, transparent spherulitic Polyzene®-F films. The resulting membranes exhibit thickness from a range from about 0.1 to about 100 μm.

### Example 7

### Preparation of Polyzene®-F Membranes by Phase-Separation Method—Evaporation of a Volatile Solvent from a Homogeneous Solution of Two or More Components

In one embodiment, Polyzene®-F membranes can be prepared by phase separation method, as follows. Exemplary Formulations: a) Deposition of a two component solvent-cast Polyzene®-F membrane from a 2 (w/v) % Polyzene®-F solution in Acetone by slow solvent evaporation at ambient temperature; and b) deposition of a two component solvent-cast Polyzene®-F membrane from a 2 (w/v) % Polyzene®-F solution in Ethyl acetate by slow solvent evaporation at ambient temperature. Membranes prepared by this method usually form opaque films with porous to fibrous or spherulitic, non-porous character. The resulting membranes exhibit thickness from a range from about 0.1 to about 100 μm.

### Example 8

### Preparation of Polyzene®-F Membranes by Phase-Separation Method—Evaporation of a Volatile Solvent from a Homogeneous Solution of Two or More Components

In one embodiment, Polyzene®-F membranes can be prepared by phase separation method, as follows. Three component mixtures tested: Polyzene®-F /Acetone/Isopropanol; and Polyzene®-F/Ethyl acetate/Isopropanol. Concentration ranges tested is 0.5-2 (w/v) % Polyzene®-F/Solvent, or any other suitable solvent/nonsolvent mixture. Membranes prepared by this method usually can form opaque films with porous to fibrous character. The resulting membranes exhibit thickness from a range from about 0.1 to about 100 μm.

Other exemplary Formulations: a) Deposition of a three component solvent-cast Polyzene®-F membrane from a 2 (w/v) % Polyzene®-F solution in a 15:85 (v/v) IprOH/EtOAc nonsolvent/solvent mixture by slow evaporation at ambient temperatures; and b) Deposition of a three component solvent-cast Polyzene®-F membrane from a 2 (w/v) % Polyzene®-F solution in a 2 (w/v) % Polyzene®-F in 20:80 (v/v) IprOH/Acetone nonsolvent/solvent mixture by slow evaporation at ambient temperatures. The resulting membranes exhibit thickness from a range from about 03.1 to about 100 μm.

### Example 9

### Preparation of Polyzene®-F Membranes by Phase-Separation Method—Addition of a Nonsolvent or Nonsolvent Mixture to a Homogeneous Solution

In one embodiment, Polyzene®-F membranes can be prepared by phase separation method, as follows. A nonsolvent/-mixture can be added to a homogeneous Polyzene®-F solution until phase separation occurs. The nonsolvent can also be introduced in gaseous state, thereby enriching itself very slowly in the Polyzene®-F solution. Examples of solvent/nonsolvent combinations include Polyzene®-F and Acetone/water (g/l), Ethyl acetate/Ethanol or Dimethylacetamide/HCl (g). Other typical non-solvents include Methanol, Isopropanol, Diethyl ether, Hexane, and the like.

Examples of other solvent/nonsolvent combinations tested: PzF/Acetone/water (g) or (1); PzF/Ethyl acetate/Ethanol; and PzF/Dimethylacetamide/HCl (g). Concentration ranges tested: 0.5-2 (w/v) % Polyzene®-F/Solvent. Other typical nonsolvents included Methanol, Isopropanol, Diethyl ether, Hexane, and the like.

Other exemplary Formulations: a) Deposition of a solvent-cast PzF membrane from a 2 (w/v) % PzF Isoamyl acetate solution by slow evaporation in presence of a saturated water vapor atmosphere at ambient temperatures; and b) Creation of a solvent-cast PzF membrane from a 2 (w/v) % PzF Acetone solution by slow evaporation in presence of a saturated water vapor atmosphere, removal of membrane, and subsequent attachment to a substrate layer by solvent welding technique using ethyl acetate vapors.

### Example 10

### Preparation of Polyzene®-F Membranes by Phase-Separation Method—Porous Closed Cell Membranes by Rapid Solvent Evaporation

In one embodiment, Polyzene®-F membranes can be prepared by phase separation method, as follows. Voluminous membranes can be prepared by rapid expansion of volatile Polyzene®-F solvents under pressure such as Dimethyl ether or Carbon dioxide through means of spray-coating.

Exemplary Formulations: An autoclave was loaded with 1 g of Polyzene®-F and 25 g of Dimethyl ether by means of condensing it with liquid nitrogen. The saturated solution can be expanded through the coaxial nozzle described in Example 3 for subsequent spray-coating application to obtain the membrane directly on a spray target area. Alternatively the compressed gas can be vented to obtain the expanded membrane directly in the autoclave.

### Example 11

### Preparation of Polyzene®-F Membranes by Phase-Separation Method —Generation of Non-Woven, Electro-Spun Polyzene®-F Fibrous Membranes

In one embodiment, Polyzene®-F membranes can be prepared by phase separation method, as follows. Generation of nano-, meso and microporous, non-woven fibrous Polyzene®-F mats can be achieved by electro-spinning a 0.5-20 mg/ml polymer solution in ethyl acetate. An electrically charged blunt needle (1-10 kV positive potential) can be fed at a rate of 0.1-10 ml/h with the spinning solution through a syringe pump. The generated stream of fibers can be directed at a grounded target, such as a stent, an aluminium foil or any other suitable conducting target over a distance of 0-20 cm. The grounded substrate can be a mandril, a flat or curved object, such as a foil, a pad, a sponge, a foam, a net, a gauze, continuous or semi-continuous, porous and/or of any other arbitrary shape and nature. The substrate, the nozzle, or both may be moved in an arbitrary fashion relative to each other to obtain e.g., a woven or non-woven pattern or any other as desired by the particular application. The obtained membranes can either be detached from the metal substrates with a dilute acid solution to obtain free-standing membranes, or bound more firmly to the substrate in question by applying solvent vapors (‘solvent welding technique’).

### Example 12

### In Vivo Experimental Design for Testing the Efficacy of MFWDM in a Patch Form

The following experiment can be performed to test the efficacy of Polymers included in formula I. The treatment groups used evaluate efficacy can include; (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B.

For comparative studies, the treatment groups to evaluate the multi-functional wound-care dressing matrix with polymer formulation I to competitive products can include treatment groups: 1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B, and exemplary competitive products.

Mice, similar in weight and age can be anesthetized, and the skin on both sides of the animal can be created by shaving and removing the hair with clippers. After proper washing, 10-15 rectangular wound sites measuring, 7 mm×10 mm, 0.3 mm deep or a wound 5 mm in diameter can be made in the paravertebral and thoracic regions of the test animal, with a cutting edge of a blade. This technique can provide complete removal of the epidermis and most superficial dermis, leaving the epidermal appendages intact. Each wound can be assigned to one of four treatment groups, and an exemplary competitive product. Wounds can then be excised from sacrificed animals representing each treatment group, and analyzed at days 1,3,5,7 and 14. Wounds that include a sufficient but constant amount of surrounding amount of marginal skin tissue, and sufficiently deep to ensure granulation tissue can be isolated and removed. The excised tissue can then be frozen in liquid nitrogen and embedded into tissue freezing medium for histologic evaluation. The freshly excised wound tissue can be placed on a membrane and bisected with a single use scalpel. Using the appropriate compound, a cryomold can then be created and placed on dry ice, and then a mold can be placed in embedding medium and stored at −80° C. until use. Histological analysis can include the accumulation and immunohistological typing of collagen present, vascularization and rate of granulation tissue, rate of epthelialization, and rate of scar formation. RNA analysis can also be performed using samples from the wound, primer sets and RNA isolated from normal skin using the procedure outlined by Chomezynski and Sacchi (Chomezynski, P. and Sacchi, N. Single-step method of RNA isolation by acid guanidinium thicyanate-phenol-chloroform extraction. Anal. Biochem. 162, 156-159, 1987). From the RNA analysis, the expression lends for several mediators for healing can be determined including microvascular blood flow, nitric oxide synthetase, endothelin, endothelin receptor, vascular endothelial growth factor, keratinocyte growth factor, and basic fibroblast growth factor. Simple measurements of wound dimensions can be used to determine the time at which 90% of the wounds are expected to heal for each treatment group. Statistical analysis can then be performed to determine the significance between treatment groups.

### Example 13

### In Vivo Experimental Design for Testing the Efficacy of MFWDM for Treating Burn Wounds

The following experiment can be performed to test the efficacy of Polymers included in formula I. The treatment groups used evaluate efficacy can include; (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B.

For comparative studies, the treatment groups to evaluate the multi-functional wound-care dressing matrix with polymer formulation I to competitive products can include treatment groups: 1) no wound dressing, (2) multi-functional wound-care dressing matrix dressing with polymer formulation I, (3) multi-functional wound-care dressing matrix dressing with polymer formulation I, with concentration A, (4) multi-functional wound-care dressing matrix dressing with polymer formulation I, with concentration B, and exemplary competitive products. Twelve six-week year old male Sprague-Dawley rats, (250 g-275 g), can be anesthetized before removing the hair on the animals back with clippers followed by washing the skin with 95% ethanol. Brass blocks or rods (2 cm×2 cm×4 cm) can be preheated by immersion in boiling water tightly controlled at 80° C. Clearly any size or metal alloy bar can be used to create the desired area of injury. A dorsal skin fold can be elevated, and two blocks applied to opposing sides of the skin fold, as required to make burn areas, 4 cm², 6 cm² and 8 cm² in total burn area that would represent an approximately 8, 12 and 16% total body surface, calculated using Meeh's formula. The skin fold can be compressed for 15 s to deliver full thickness (class III) skin burn, 2-5 wound sites can be made in the paravertebral and thoracic regions of the animal. The use of staples to secure the outer most layer of wound dressing at the cephalad and caudad edges can be desirable, followed by wrapping to prevent the dressing from migrating with animal motion. Rats can be sacrificed at 72 hour intervals for 15 days. Biopsies of the wound site can be used to determine infection by measuring the presence of erythema, purulence, or microscopically by intradermal neutrophils containing bacteria, blood can also be used to measure infection colony counts. Reepithelialization can be observed microscopically, by measuring the length of neoepidermis at each time point to allow quantification of the degree of reepithelialization even for wounds that are not completely reepithelialized. Histopathological studies can also be done on formalin-fixed alcohol, dehydrated, xylene-cleared, paraffin-embedded, stained sections using conventional microscopy to determine rate and type of collagen, rate of vascularization of granulation tissue, clearance of bacteria from the wound, rate of contraction, and degree of scar formation. Also, a measurement device can be used to evaluate the wound dimensions at time of sacrifice to determine the time at which 90% of the wounds are expected to heal for each treatment group. Statistical analysis can then be performed to determine the significance between treatment groups.

### Example 14

In Vivo Experimental Design for Testing the Efficacy of MFWDM for Treating Ulcer Wounds

The following experiment can be performed to test the efficacy of Polymers included in formula I. The treatment groups used evaluate efficacy can include; (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B.

For comparative studies, the treatment groups to evaluate the multi-functional wound-care dressing matrix with polymer formulation I to competitive products can include treatment groups: (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B, and exemplary competitive products. Intracutaneous injection of sodium tetradecyl sulphate (STD) can be injected into the flank skin of 40 guinea-pigs, thus creating a reproducibly sized and shaped superficial ulcer. The ulcer can then be treated with dressings from the treatment groups evaluated after animal sacrifice at 5, 10, 30 and 60 days post infliction. Dressings can be secured using stapes or wrapping to prevent the dressing from migrating with animal movement. Tissue biopsies taken from test animals at sacrifice can be used to determine the histopathology of the vascularization, formation of granulation tissue, clearance of bacteria, type or organization of collagen, rate of reepithelialization, degree of scar formation, leukocyte infiltration, trasncutaneous oxygen tension and blood flow to the wound and proximal wound tissue. RNA analysis can also be performed using samples from the wound, primer sets and RNA isolated from normal skin using the procedure outlined by Chomezynski and Sacchi (Chomezynski, P. and Sacchi, N. Single-step method of RNA isolation by acid guanidinium thicyanate-phenol-chloroform extraction. Anal. Biochem. 162, 156-159, 1987). From the RNA analysis, the expression lends for several mediators for healing can be determined including microvascular blood flow, nitric oxide synthetase, endothelin, endothelin receptor, vascular endothelial growth factor, keratinocyte growth factor, and basic fibroblast growth factor. Simple measurements of wound dimensions can be used to determine the time at which 90% of the wounds are expected to heal for each treatment group. Statistical analysis can then be performed to determine the significance between treatment groups.

### Example 15

### In Vivo Experimental Design for Testing the Efficacy of MFWDM for Treating Severe-Deep Tissue Wounds

The following experiment can be performed to test the efficacy of Polymers included in formula I. The treatment groups used evaluate efficacy can include; (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B.

For comparative studies, the treatment groups to evaluate the multi-functional wound-care dressing matrix with polymer formulation I to competitive products can include treatment groups: (1) no wound dressing, (2) multi-functional wound-care dressing matrix dressing with polymer formulation I, (3) multi-functional wound-care dressing matrix dressing with polymer formulation I, with concentration A, (4) multi-functional wound-care dressing matrix dressing with polymer formulation I, with concentration B, and exemplary competitive products. Eight, white domestic young pigs can be anesthetized, and the wounds on both sides of the animal can be created by removing the hair with clippers and washing with 95% ethanol. After proper washing, 30-50 rectangular wound sites measuring, 7 mm×10 mm, 1.0 mm deep can be made in the paravertebral and thoracic regions of the test animal with a cutting edge of a blade. A set of wounds can then be excised from sacrificed animals and from each treatment group, and analyzed at days 1,5,10 and 20. Wounds that include a a sufficient but constant amount of surrounding amount of marginal skin tissue, and deep enough to ensure granulation tissue can be isolated and removed. The excised tissue can then be frozen in liquid nitrogen and embedded into tissue freezing medium for histology. The freshly excised wound tissue can be placed on a membrane and bisected with a single use scalpel. Using the appropriate compound, a cryomold can then be created and placed in embedding medium and stored at −80° C. until use. Histological analysis can include the accumulation and immunohistological typing of collagen present, vascularization and rate of granulation tissue, rate of epthelialization, transcutaneous oxygen tension, tissue necrosis, and rate of scar formation. RNA analysis can also be performed using samples from the wound, primer sets and RNA isolated from normal skin using the procedure outlined by Chomezynski and Sacchi (Chomezynski, P. and Sacchi, N. Single-step method of RNA isolation by acid guanidinium thicyanate-phenol-chloroform extraction. Anal. Biochem. 162, 156-159, 1987). From the RNA analysis, the expression lends for several mediators for healing can be determined, including microvascular blood flow, nitric oxide synthetase, endothelin, endothelin receptor, vascular endothelial growth factor, keratinocyte growth factor, and basic fibroblast growth factor. Simple measurements of wound dimensions can be used to determine the time at which 90% of the wounds are expected to heal for each treatment group. Statistical analysis can then be performed to determine the significance between treatment groups.

### Example 16

### In Vivo Experimental Design for Testing the Efficacy of MFWDM for Treating Necrotic Tissue Wounds

The following experiment can be performed to test the efficacy of Polymers included in formula I. The treatment groups used evaluate efficacy can include; (1) no wound dressing, (2) multi-functional wound-care dressing matrix dressing without polymer formulation I, (3) multi-functional wound-care dressing matrix dressing with polymer formulation I, with concentration A, (4) multi-functional wound-care dressing matrix dressing with polymer formulation I, with concentration B.

For comparative studies, the treatment groups to evaluate the multi-functional wound-care dressing matrix with polymer formulation I to competitive products can include treatment groups: (1) no wound dressing, (2) multi-functional wound-care dressing matrix dressing with polymer formulation I, (3) multi-functional wound-care dressing matrix dressing with polymer formulation I, with concentration A, (4) multi-functional wound-care dressing matrix dressing with polymer formulation I, with concentration B, and exemplary competitive products. Two, specific-pathogen-free female white Yorkshire pigs (12-15 kg) can be anesthetized, the hair removed from the dorsal skin of the paravertebral and thoracic regions, and the exposed skin washed with 95% ethanol. Twenty one full-thickness incisional wounds created with a 6 mm biopsy punch (0.5 mm deep) can be created. A biopsy can be taken from the control group to provide a baseline for assessing initial wound parameters. Wounds can be covered with dressings from the treatment groups, and on days 2 and 4 gently cleansed, and dressings reapplied. Analysis can be performed on days 5 and 10 after sacrifice. Wounds can be evaluated clinically by macroscopic inspection for fluid accumulation, appearance of surrounding normal skin, presence of debrided tissue, debridement of wound eschar, and punctuate bleeding. The biopsies removed from all wounds providing a deep cross-section of the wounds can be fixed, mounted, and stained with hematoxylin, eosin and elastochrome. Sections can then be evaluated microscopically for indicators of debridement and healing. These can include debridement of necrotic eschar or fibrinous clot, epidermal migration and maturation, inflammatory cells, extracellular matrix, new blood vessels and a global assessment of healing. Scores can be assigned either on an absolute scale or relative to the untreated control for each treatment group. Statistical analysis can then be performed to determine the significance between treatment groups.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalent.

We claim:

1. A multi-functional wound-care dressing matrix, comprising:
a substrate layer formed as a wound dressing comprising at least one tissue-contacting surface incorporating
at least one high molecular weight polyphosphazene polymer of formula (I): 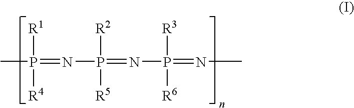
wherein
n is an integer from about 40 to about 100,000;
R¹ to R⁶ are independently selected from:
a) a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
b) a heterocyclic alkyl group with at least one nitrogen, phosphorus, oxygen, sulfur, or selenium as a heteroatom;
c) a heteroaryl group with at least one nitrogen, phosphorus, oxygen, sulfur, or selenium as the heteroatom;
d) a nucleotide or a nucleotide residue;
e) a biomacromolecule; or
f) a pyrimidine or a purine base.

2. The multi-functional wound-care dressing matrix of claim 1, wherein at least one R¹ to R⁶ substituent is an alkoxy group substituted with at least one fluorine atom.

3. The multi-functional wound-care dressing matrix of claim 1, wherein at least one R¹ to R⁶ substituent is selected from the group consisting of: OCH₃, OCH₂CH₃, O(CH₂)₂CH₃, O(CH₂)₃CH₃, O(CH₂)₄CH₃, O(CH₂)₅CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, and OCH₂(CF₂)₇CHF₂.

4. The multi-functional wound-care dressing matrix of claim 1, wherein the R¹ to R⁶ substituent is 1% or less of an alkenoxy group.

5. The multi-functional wound-care dressing matrix of claim 1, wherein the polyphosphazene polymer of formula (I) has a molecular weight of at least 2,000,000 g/mol.

6. The multi-functional wound-care dressing matrix of claim 1, wherein the polyphosphazene polymer of formula (I) forms a coating over at least the tissue-contacting surface of the substrate layer, partially or entirely.

7. The multi-functional wound-care dressing matrix of claim 1, wherein the substrate layer comprises one or more of the following: synthetic polymers, polymer blends, block polymers, blends of block polymer, naturally occurring plant-derived materials, modified plant-derived materials, and modified animal-derived materials.

8. The multi-functional wound-care dressing matrix of claim 1, wherein the substrate layer is selected from the group consisting of: a woven fabric layer, a non-woven fabric layer, a porous film, a non-porous film, a porous membrane, a non-porous membrane, an open-cell foam, a closed-cell foam, a woven mat, a non-woven mat, a mesh, a pad, a sponge, a foam, a sponge, and a gauze.

9. The multi-functional wound-care dressing matrix of claim 1, wherein the substrate layer further comprises capsules comprising agents of interest.

10. The multi-functional wound-care dressing matrix of claim 9, wherein the capsules have an average diameter size ranging from approximately 10 μm to approximately 1200 μm.

11. The multi-functional wound-care dressing matrix of claim 1, wherein the substrate layer is a foamed sponge.

12. The multi-functional wound-care dressing matrix of claim 11, further comprises a tubing member that removes excess bodily fluids from the tissue-contacting surface of the substrate layer.

13. The multi-functional wound-care dressing matrix of claim 1, wherein the substrate layer contacts a wound site selected from the group consisting of: cuts, gashes, open wounds, tissue rupture, Decubitus, Dermatitis, lesions, chronic wounds, battlefield wounds, necrotic wounds, acute, chronic, traumatic, lacerations, abrasions, contusions, necrotizing facitis, toxic epidermal nercolysis, pressure wounds, venous insufficiency ulcers, arterial ulcers, diabetic ulcer, neuropathic ulcers, pressure ulcers, mixed ulcers, burn wounds, Mucormycosis, Vasculitic wounds, and Pyoderma, gangrenosum,

14. The multi-functional wound-care dressing matrix of claim 1 further comprises one or more superimposed substrate layers that are deposited above the first substrate layer when oriented with respect to a wound site, wherein the materials are selected from comprises one or more of the following: synthetic polymers, polymer blends, block polymers, blends of block polymer, naturally occurring plant-derived materials, modified plant-derived materials, and modified animal-derived materials.

15. The multi-functional wound-care dressing matrix of claim 14, wherein the superimposed substrate layer is selected from the group consisting of: a woven fabric layer, a non-woven fabric layer, a porous film, a non-porous film, a porous membrane, a non-porous membrane, an open-cell foam, a closed-cell foam, a woven mat, a non-woven mat, a mesh, a pad, a sponge, a foam, a sponge, and a gauze.

16. The multi-functional wound-care dressing matrix of claim 14, wherein the substrate layer is a foamed sponge.

17. The multi-functional wound-care dressing matrix of claim 16, further comprises a tubing member that removes excess bodily fluids from the tissue-contacting surface of the substrate layer.

18. A method for producing multi-functional wound-care dressing matrix, comprising:
incorporating onto at least one tissue-contacting surface of a substrate layer formed as a wound dressing,
at least one high molecular weight polyphosphazene polymer of formula (I): 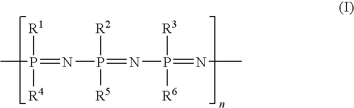
wherein
n is an integer from about 40 to about 100,000;
R¹ to R⁶ are independently selected from:
a) a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
b) a heterocyclic alkyl group with at least one nitrogen, phosphorus, oxygen, sulfur, or selenium as a heteroatom;
c) a heteroaryl group with at least one nitrogen, phosphorus, oxygen, sulfur, or selenium as the heteroatom;
d) a nucleotide or a nucleotide residue;
e) a biomacromolecule; or
f) a pyrimidine or a purine base.

19. A method for healing wounds, the method comprising:
covering a wound site with a multi-functional wound-care dressing matrix, comprising:
a substrate layer formed as a wound dressing comprising at least one tissue-contacting surface incorporating
at least one high molecular weight polyphosphazene polymer of formula (I): 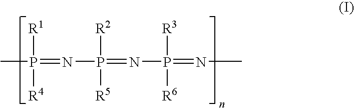
wherein
n is an integer from about 40 to about 100,000;
R¹ to R⁶ are independently selected from:
a) a substituted or unsubstituted alkyl, alkoxy, aryl, aryloxy, silyl, silyloxy, alkylsulfonyl, alkyl amino, dialkyl amino, ureido, carboxylic acid ester, alkylmonoamidine, alkylbisamidine, alkoxymonoamidine, or alkoxybisamidine; or an amino;
b) a heterocyclic alkyl group with at least one nitrogen, phosphorus, oxygen, sulfur, or selenium as a heteroatom;
c) a heteroaryl group with at least one nitrogen, phosphorus, oxygen, sulfur, or selenium as the heteroatom;
d) a nucleotide or a nucleotide residue;
e) a biomacromolecule; or
f) a pyrimidine or a purine base; and
permitting a sufficient time for the wound to heal.
